(19) United States
(12) Patent Application Publication
(10) Pub. No.: US 2022/0091110 A1
(43) Pub. Date: Mar. 24, 2022

(54) Chemically Amplified Detection

(71) Applicant: Joseph R. Hunt, King George, VA (US)

(72) Inventor: Joseph R. Hunt, King George, VA (US)

(73) Assignee: United States of America, as represented by the Secretary of the Navy, Arlington, VA (US)

(21) Appl. No.: 17/027,970

(22) Filed: Sep. 22, 2020

(51) Int. Cl. G01N 33/543

(52) U.S. Cl. CPC G01N 33/54306; G01N 33/5432

(57) ABSTRACT

A method is provided for detecting a biological analyte in a medium. The method includes enclosing a catalyst in an encapsulate that includes an associated binder; adding reactants to the medium, followed by adding the encapsulate that contains the encapsulate to the medium. In response to presence of the analyte in the medium, the binder attaches to the analyte and opens the encapsulate to release the catalyst. The catalyst accelerates chemical change of the reactants into products. The method continues by detecting the products to register presence of the analyte.

### STATEMENT OF GOVERNMENT INTEREST

The invention described was made in the performance of official duties by one or more employees of the Department of the Navy, and thus, the invention herein may be manufactured, used or licensed by or for the Government of the United States of America for governmental purposes without the payment of any royalties thereon or therefor.

### BACKGROUND

The invention relates generally to detection of biological agents. In particular, the invention employs reactants in a medium with encapsulates to release catalysts in presence of such biological agents to produce surrogate products from the reactants, with such products being easier to detect than the agents.

Analytical methods to detect chemical and biological analytes is well established, although trade-offs are involved in these techniques. In order to detect very small amounts of an analyte, complicated, large, and expensive instruments are usually required because they have the high sensitivity to detect very small amounts.

Detection with smaller hand held instruments is possible, but usually requires the amplification of the analyte signal. This is especially valid with biological detection. Such detection typically employs the polymerase chain reaction (PCR) to amplify specific biological signatures to a concentration within the detection range of the instruments. The problem is that this PCR process is expensive, time consuming, and requires special chemicals and equipment. Biological assays are also used in bio-detection, but these assays lack high sensitivity and sometimes involve ambiguous results such as color changing reactions. One potential way to implement this chemical amplification technology would be using immunoliposomes.

Biological encapsulants such as immunoliposomes can implement this chemical amplification. Such immunoliposomes are produced from the self assembly of lipid bilayers. Antibody receptors or recognition sites can be incorporated onto the surface of the liposomes. When the target for the recognition site binds, the liposomes can start to come apart. If the liposomes are formed in the presence of a desired species, then that species can be trapped inside the liposome. When the binding occurs and the liposome breaks open the species can be released.

Conventional chemical detectors can rapidly detect different chemicals in the environment or air. These instruments include toxic gas detectors used in industrial applications, chemical agent detectors, or analytical equipment used in laboratories. Current options for real time biological detection are lacking, as conventional processes involve from 30 minutes up to four hours and have low sensitivities.

### SUMMARY

Conventional biological detection methods yield disadvantages addressed by various exemplary embodiments of the present invention. In particular, exemplary embodiments provide a method for detecting a biological analyte in a medium. The method includes enclosing a catalyst in an encapsulate that includes an associated binder; adding reactants to the medium, followed by adding the encapsulate that contains the encapsulate to the medium.

In response to presence of the analyte in the medium, the binder attaches to the analyte and opens the encapsulate to release the catalyst. The catalyst accelerates chemical change of the reactants into products. The method continues by detecting the products to register presence of the analyte. In various exemplary embodiments, the encapsulate is a liposome.

### BRIEF DESCRIPTION OF THE DRAWINGS

These and various other features and aspects of various exemplary embodiments will be readily understood with reference to the following detailed description taken in conjunction with the accompanying drawings, in which like or similar numbers are used throughout, and in which:

FIG. 1 is a schematic view of a chemical detection process.

### DETAILED DESCRIPTION

In the following detailed description of exemplary embodiments of the invention, reference is made to the accompanying drawings that form a part hereof, and in which is shown by way of illustration specific exemplary embodiments in which the invention may be practiced. These embodiments are described in sufficient detail to enable those skilled in the art to practice the invention. Other embodiments may be utilized, and logical, mechanical, and other changes may be made without departing from the spirit or scope of the present invention. The following detailed description is, therefore, not to be taken in a limiting sense, and the scope of the present invention is defined only by the appended claims.

The objective of this disclosure is to describe the chemical amplification concept and its use to enhance the sensitivity of detection systems. This liposome binding process is already well established. However, this can be applied to exemplary process embodiments. These processes amplify a chemical signal using the surrogate production of a specific reaction product due to the triggered release of a catalyst for the purposes of improved sensitivity in detection applications. One potential example of this exemplary technology involves immunoliposomes.

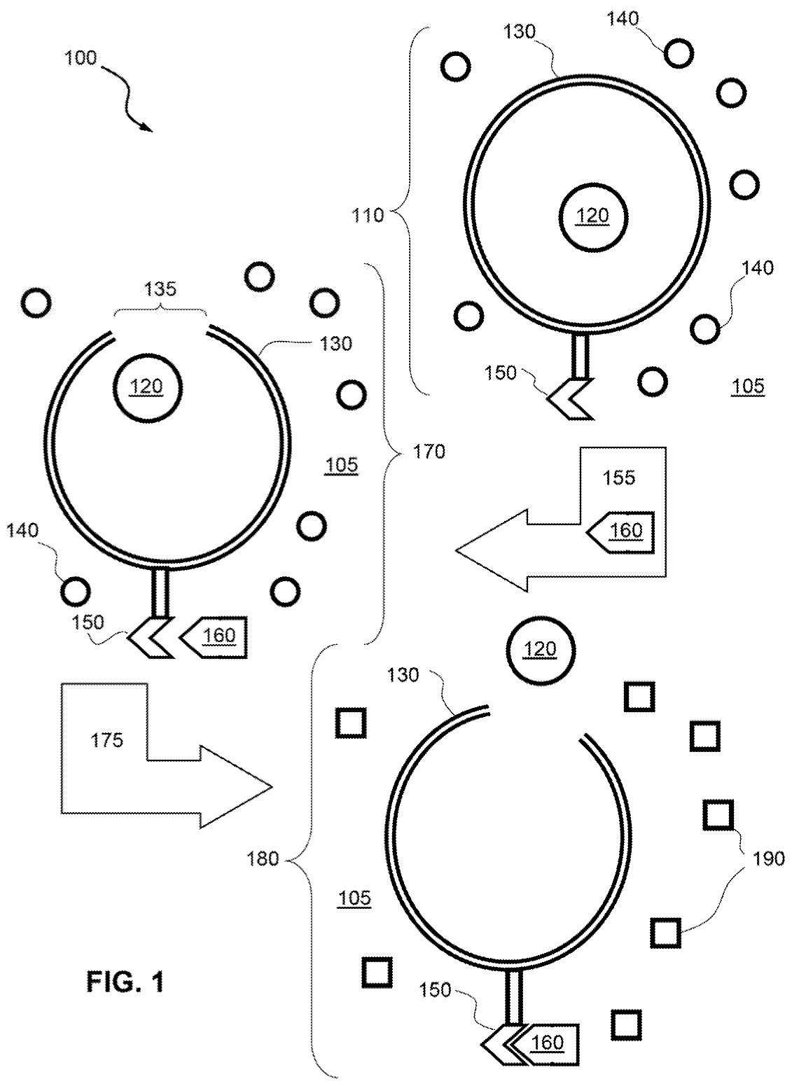

FIG. 1 shows a schematic view 100 of the exemplary reaction in a medium 105. In a precursor scenario 110, a catalyst 120 is enveloped by an encapsulant 130 that can subsequently produce an opening 135. Several reagents 140 surround the encapsulant 130 to which a binder 150 (e.g., an antibody) attaches. A first transition 155 introduces an analyte 160 (e.g., a biological threat) into the medium 105 to a transition scenario 160. In response to the analyte 160, the encapsulant 130 separates ends to form the opening 135. This condition yields a second transition 175 to a post reaction scenario 180 for discharging the catalyst 120. The released catalyst 120 accelerates chemical reaction in the medium 105 of the reactants 140 into products 190.

Thus, view 100 illustrates the exemplary chemical detection amplification process showing conversion of reagents 140 to products 190 after release of catalyst 120. This process involves disposing a catalyst 120 inside an encapsulant 130, such as a liposome or immunoliposome, and inserting the encapsulant 130 into an environment medium 105 containing a reagent 140 that the catalyst 120 can react with to produce the product 190. The reagents 140 can be any organic reagent that can be chemically transformed, while the products 190 can be the product of that chemical transformation. The medium 105 can for example be water or any organic solvent or medium in which the encapsulant 160 is stable. The chemical reaction and catalyst of choice for encapsulation would depend on the detection means being used.

For employment of instrumentation such as Fourier transform infrared spectroscopy, nuclear magnetic resonances spectroscopy or mass spectrometry, then the reaction could be a simple organic functional group transformation where detection of the product would be through chemical analysis. This could include reactions such as (a) an esterification (i.e., a reaction to produce an ester) where a lewis acid catalyst would be needed to react with a carboxylic acid (C(═O)OH) and an alcohol, or else (b) a reduction reaction where a reducing agent such as sodium borohydride (NaBH₄) were needed to react with an aldehyde (—CHO) or carboxcylic acid. The detection could also be accomplished through visual means where a color change or change in fluorescent properties is indicative of release of the catalyst 120 from the encapsulant 130, including catalyzed reactions that produce fluorescent dyes.

The surface of the encapsulant 130 breaks to interact with the desired analyte 160, whether chemical or biological. This analyte 160 could involve antibodies in the biological detection case where immunoliposomes have antibodies as the binders 150 on the surface for specific biological species. A chemical or biological detection target (as analyte 160) binding to the immunoliposome (as encapsulant 130) via the antibody (as binder 150) releases the catalyst 120. This enables the catalyst 120 to accelerate transformation of the surrounding reagent 140 to a known product 190.

The detection of this product 190 can then be used as a surrogate to determine the presence of analyte 160 as the target species for biological detection. The concept could easily be demonstrated by utilizing immunoliposomes that contain a simple enzyme, such as horseradish peroxidase (a large alpha-helical protein that binds heme as a redox cofactor), or else an enzyme that catalyzes the production of fluorescent species. The release and subsequent detection of color change or fluorescence could be used to demonstrate proof of concept. The analyte 160 as target could be a common bacteria like E. coli. Such bacteria have been evaluated in the literature. For example, see M. G. Beeman et al. “Electrochemical Detection of E. coli O157:H7 in Water after Electrocatalytic and Ultraviolet Treatments . . . ” Sensors 18(1497) (2018, https://ww.nchi.nlm.nih.gov/pmc/articles/PMC5981196/pdf/sensors-18-01497.pdf)

The exemplary process can be summarized by the following operations: First, enclose catalysts 120 in corresponding encapsulates 130 that include associated binders 150. Second, prepare a medium 105 for evaluation. Third, add reactants 140 to the medium 105. Fourth, add the catalyst-containing encapsulates 130 to the medium 105. Fifth, in response to an analyte 160 being present in the medium 105, one of the binders 150 attaches to the analyte 160 and opens the associated encapsulate 130 to release the corresponding catalyst 120, which accelerates chemical change of the reactants 140 into products 190. Sixth, these products 190 are detected by a sensor, which registers the presence of the analyte 160 as the target substance.

The commercial potential for example embodiments lies in its ability to amplify a signal from very low levels of analyte 160. The release of the catalyst 120 could be triggered by a single interaction of a medium 105 with encapsulation 130 and one analyte 160 as the target. That single release of catalyst 120 can then trigger the initiation of the total conversion of the reagent 140 present in the medium 105. This indicates that only one interaction is necessary to trigger the detection of targeted species, vastly increasing system sensitivity. This is far superior to simple biological and chemical assays in which multiple bacteria interactions are required to produce a noticeable color change, and this sometimes produces ambiguous results.

The commercial potential in biological detection would involve improved detectors that can rapidly detect very small concentrations of bacteria in real time. Conventional methods require immunoassays, which offer ambiguous results and require tens of minutes, or else PCR based detection methods, which are accurate but require one-to-four hours to obtain confirmatory results. Exemplary embodiments of this process could be incorporated into conventional systems to produce much faster and more sensitive detectors. I. A. Darwish provides further information in “Immunoassay Methods and their Applications in Pharmaceutical Analysis”, Int. J. of Biomed. Sci. 2(3), 217-235 (2006, available at https://www.ncbi.nlm.nih.gov/pmc/articles/PMC3614608/pdf/IJBS2-217.pdf).

The broader commercial potential would involve applying this concept to detection methods to enhance sensitivity and render detection cheaper. Typically as previously mentioned, high sensitivity requires large, expensive instrumentation. The exemplary process enhances the signal of an analyte 160 sufficiently so that cheaper less sensitive instruments could be used for detection. Using a small, cheap handheld detector in field would now be possible because this would be detecting the catalyzed product and not the small target concentration.

The goal of this exemplary process was mainly developed to provide rapid, real time biological detection. Detecting the presence of chemicals in real time is currently possible, although this concept could improve the sensitivity of those systems as well. Real time detection of biological species is conventionally unavailable, and this exemplary concept can be used to satisfy this unmet challenge.

The advantages of exemplary embodiments are versatility, sensitivity, and selectivity. The concept is versatile because any encapsulant 130 or catalyst 120 could be utilized depending on the application, including different detection methods (color change, fluorescence, analytical detection), recognition methods (assays, surface proteins, chemical functional groups), or encapsulants 130. The concept provides high sensitivity due to the amplification in response to a single binding event and selectivity could be determined by the choice of recognition method.

While certain features of the embodiments of the invention have been illustrated as described herein, many modifications, substitutions, changes and equivalents will now occur to those skilled in the art. It is, therefore, to be understood that the appended claims are intended to cover all such modifications and changes as fall within the true spirit of the embodiments.

What is claimed is:

1. A method for detecting a biological analyte in a medium, said method comprising:
enclosing a catalyst in an encapsulate that includes an associated binder;
adding reactants to the medium;
adding said encapsulate that contains said encapsulate to the medium, wherein
in response to presence of the analyte in the medium, said binder attaches to the analyte and opens said encapsulate to release said catalyst, which accelerates chemical change of said reactants into products; and
detecting said products to register presence of the analyte.

2. The method according to claim 1, wherein said encapsulate is a liposome.

3. The method according to claim 1, wherein said encapsulate is a immunoliposome.

4. The method according to claim 1, wherein the medium is water.

5. The method according to claim 4, wherein said reactants include caboxcylic acid and said products include an ester.
